(19) United States
(12) Patent Application Publication
(10) Pub. No.: US 2004/0143280 A1
(43) Pub. Date: Jul. 22, 2004

(54) Flexible wire transection the transverse carpal ligament

(76) Inventor: Loubert Suddaby, Orchard Park, NY (US)

Correspondence Address: SHOEMAKER AND MATTARE, LTD, 10 POST OFFICE ROAD - SUITE 110, SILVER SPRING, MD, 20910, US

(21) Appl. No.: 10/346,181

(22) Filed: Jan. 17, 2003

(51) Int. Cl. A61B 17/32

(52) U.S. Cl. 606/167

(57) ABSTRACT

A flexible cutting filament or wire is led through the carpal tunnel, beneath the transverse carpal ligament, by a passer. Thereafter, the opposite ends of the wire are secured in an instrument which tightens the wire and may be used to move the wire as a cutting tool to transect the overlying ligament, while preserving surrounding tissues. The small wire diameter enables minimally invasive techniques to limit post-operative pain and speed recovery.

### BACKGROUND OF THE INVENTION

This invention relates to surgery, and more particularly to percutaneous or endoscopic transection of the transverse carpal ligament.

First described in 1933, carpal tunnel release surgery is now considered the most frequently performed operation in the United States. Although carpal tunnel syndrome can be caused by a variety of clinical disorders (hypothyroidism, diabetes, pregnancy, etc.), occupational injury or repetitive strain syndrome is now the most frequent association. Indeed, carpal tunnel syndrome is second only to back injuries as the most common reason for employee absenteeism. With repetitive use of the hand, the transverse carpal ligament is thought to hypertrophy thereby compressing the median nerve running beneath it and causing the compression neuropathy known as carpal tunnel syndrome.

The carpal tunnel is formed dorsally by the proximal row of carpal bones. Ventrally, the broad ligament known as the transverse carpal ligament extends from the hook of the hamate bone medially to the trapezium bone laterally to form the roof or ventral boundary of the carpal tunnel. Within the tunnel pass the flexor tendons of the hand, the median nerve and associated synovial tissues associated with the flexor tendons.

While a variety of temporizing measures can be used to treat the condition (splinting, anti-inflammatory medication, steroid injection), only surgery is considered curative. Because surgery for this condition enjoys a very high success rate with low morbidity, it is frequently chosen as the definitive treatment option.

The surgical treatment of this condition can be broadly divided into two types: open versus endoscopic.

With the open procedure, the skin lying over the carpal tunnel is incised and the transverse carpal ligament is then transected under direct vision. The skin is then reapproximated with sutures.

In the endoscopic version, small portals are made in the skin and the transverse carpal ligament is transected endoscopically without major disruption of the overlying skin and subcutaneous tissues. Because the majority of pain receptors are located in the skin, limiting surgical trauma to the ligament results in significantly less pain attributable to the procedure, and a shorter convalescent period.

Since endoscopic procedures involve smaller skin incisions as compared to the open procedures, they are favored by many surgeons in the treatment of this condition. Present endoscopic procedures require passing an endoscope and associated cutting instruments through the carpal tunnel to facilitate the endoscopic operation. In severe forms of carpal tunnel syndrome, the hypertrophied transverse carpal ligament renders the carpal tunnel quite narrow. Indeed, this is the pathologic process by which the median nerve becomes compressed. When the carpal tunnel is narrow, it becomes difficult and sometimes impossible to pass all of the necessary equipment needed to perform the release surgery. This occurs because the endoscope and associated instruments have a fixed diameter which the pathologically narrow carpal tunnel may not be able to accommodate. For this reason, fully 15% to 20% of endoscopic procedures cannot be completed and must be converted to open procedures. In addition, even in successful endoscopic procedures, significant paresthesia may be noted post-operatively because of damage to the median nerve that occurs when surgical endoscopes and instruments are passed through a pathologically narrow carpal tunnel.

### SUMMARY OF THE INVENTION

This invention relates to a method and device by which the transverse carpal ligament can be transected either endoscopically, or percutaneously without an endoscope, utilizing instruments which present the smallest cross-sectional area thereby allowing surgery to be performed even in extremely tight or narrowed carpal tunnels.

To facilitate this, a flexible cutting instrument, for example a wire, is passed through a proximal skin portal and is retrieved through a skin portal which is distal to the transverse carpal ligament. The term “wire” as used herein should be understood to mean not only metal wire, but also thin rod, string, cord, polymeric filament, and the like made materials having sufficient strength to be effective. After the wire is in place, an instrument having a pair of spaced pillars is applied to the hand so that each pillar is located at one of the portals. When the wire is drawn taut, it runs parallel to the body of the bridge, and the intervening skin remains intact while the wire cuts the ligament. The wire and bridge can then be disassociated and the wire removed.

In a uniportal technique utilizing this same concept, the flexible cutting instrument or wire is fixed at one end of a spatula-shaped passer. The spatula is jointed, or at least sufficiently flexible that it will bend when the wire is tautened. This flexibility allows the wire to disassociate from the passer along its length except at its terminal attachment point. The instrument thereby takes on the configuration of a bow with the cutting wire approximating a bow string and the spatula passing component forming the curved limbs of the bow. The bow assembly can then be manipulated to and from when the wire is tautened and thereby cut through the overlying ligament.

The wire can be passed via a flat or spatula-shaped passer if the percutaneous method is chosen, or via a cylindrical sheath designed to fit over any commercially available endoscope, if the endoscopic method is chosen. In either case, the wire is embedded or affixed in a groove or channel in the spatula passer or cylindrical sheath and can be readily disengaged from the passer or sheath when tautened against the overlying ligament. This is the case in either the biportal or uniportal technique.

The advantage of this new procedure over present percutaneous or endoscopic methods is that the diameter of the wire used to cut the ligament can be much smaller than the blades presently used to percutaneously transect the transverse carpal ligament. In addition, because the flexible cutting instrument and passer are of limited cross-sectional size, smaller skin access portals can be used. The method described is also simpler, requires less costly materials, and can potentially be done without an expensive endoscope. Additionally, by using different pillar depths in the biportal technique, variations on the degree of transection of the ligament can be achieved so that partial depth transection of the ligament may be performed. This allows enlargement of the carpal tunnel while still preserving the functional integrity of the ligament itself, which serves as the stabilizing structure for the origin of the abductor pollicus brevis and the abductor digiti minimi.

A principal object of the present invention is to improve the present method of percutaneous and endoscopic carpal tunnel surgical release techniques. This invention provides for transection of the transverse carpal ligament by a flexible element (string or wire) made of metal or any variety of synthetic materials having sufficient tensile strength when drawn into small diameters to have tissue cutting properties when drawn taut. The string or wire may be smooth or corrugated. In the corrugated embodiment it can function as a saw when manipulated to and fro along its length.

Because of its flexibility and small cross-sectional diameter, the flexible-element cutting instrument can be more easily passed through the carpal tunnel than prior cutting instruments. Presently-used cutting instruments generally approximate the form of a cutting blade or hook and thereby necessitate larger cross-sectional areas by virtue of their blade or hook status or by the accompanying instruments necessary to manipulate them safely within the carpal tunnel.

In biportal version this invention, the instrument manipulating the flexible cutting device (bridge assembly system) lies outside the confines of the carpal tunnel (external to the skin), thereby minimizing the diameter and number of surgical instruments within the tunnel while the surgery is being performed. In the uniportal technique, the passer becomes the manipulating instrument, but it is of such a small diameter that its presence within the carpal tunnel poses no detriment to the median nerve and serves to displace the nerve away form the cutting wire.

The present invention can be employed with or entirely without an expensive endoscope, thereby providing the added benefit of cost containment, while still allowing for individual surgeon preference.

### BRIEF DESCRIPTION OF THE DRAWINGS

In the accompanying drawings,

FIGS. 1a-1c show a wire passer used to pass a cutting wire between distal and proximal portals in the skin;

FIG. 2a shows in simplified form a cross-section of the transverse carpal ligament beneath the skin, and FIGS. 2b-2e show how the passer is used to thread a cutting wire the carpal tunnel;

FIG. 3 shows a wire tightening instrument in detail;

FIG. 4 shows the instrument applied between the portals;

FIG. 5a is a view like FIG. 2a, showing the situation of FIG. 4;

FIG. 5b demonstrates the ends of the wires being drawn up around the ends of the instrument and wound around the shaft of its knob;

FIG. 5c shows the knob being turned to tauten the wire; and

FIG. 5d shows the position of the wire once the ligament has been transected.

FIGS. 6a-6d show and alternative embodiment of the invention, where a spatula-type device is used as both a wire passer and a wire manipulator for transection.

### DESCRIPTION OF THE PREFERRED EMBODIMENT

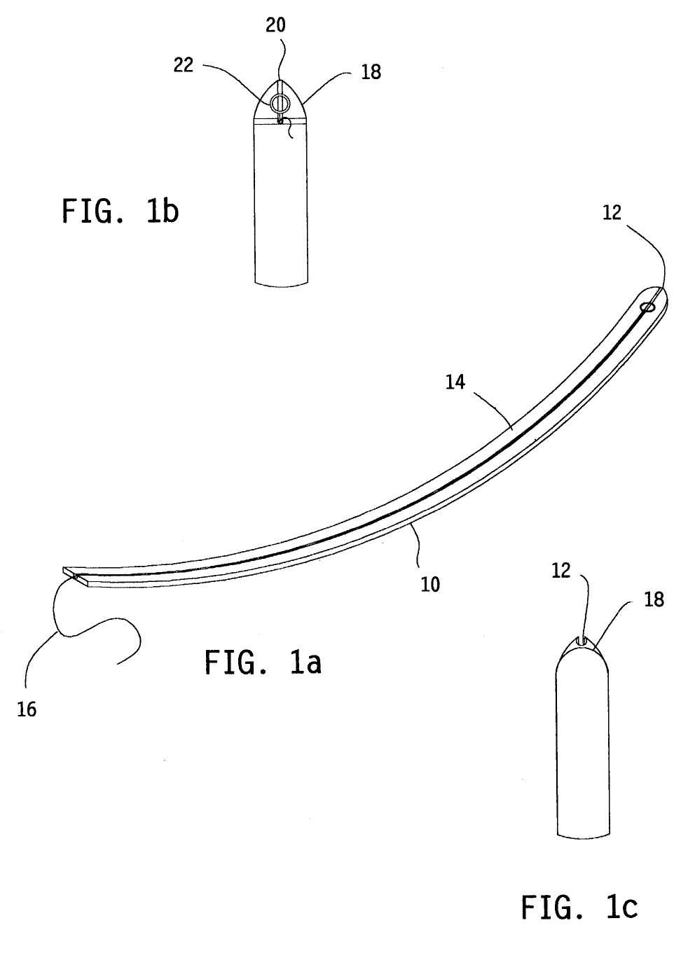

FIGS. 1a-1c show a curved wire passer 10, which has a slot 12 running lengthwise in one of its broad faces 14, for receiving and holding a cutting wire 16. While a flat spatula-shape is presently preferred, the passer could alternatively be tubular, to allow placement over an endoscope.

The passer has a large-radius curvature corresponding to the intended path of the wire beneath the ligament. It may be rigid, or flexible but with enough rigidity so that it can be pushed through the carpal tunnel. The nose 18 is rounded, tapering to a radiused tip 20. A clamp or notch 22 (FIG. 1b) at the nose holds the distal end of the wire initially.

The wire 16 may be metallic, or formed from another suitable material having sufficiently high tensile strength and hardness to cut through the transverse carpal ligament. It may have a uniform circular cross-section, or it may be formed with serrations, corrugations or other irregularities to improve its cutting action. The wire is pressed into the slot 12, sized to retain the wire while the wire is being passed through the carpal tunnel, so as to prevent the wire from straying and possibly injuring adjoining tissues.

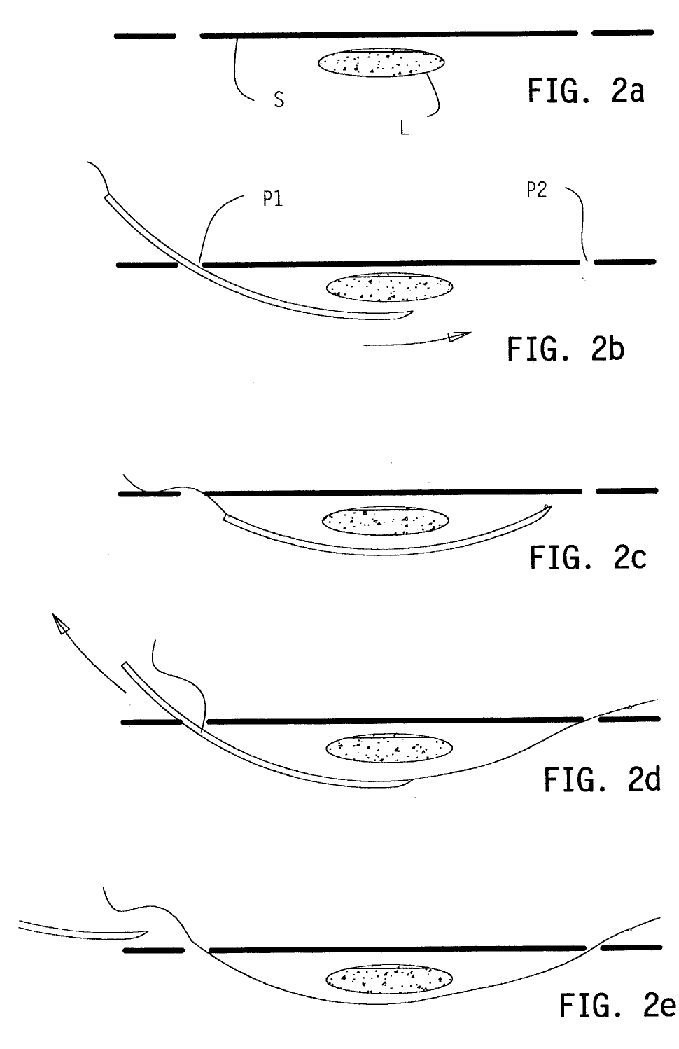

FIG. 2a shows, in simplified form, the transverse carpal ligament “L” beneath the skin “S” of the wrist.

In FIG. 2b, the passer has been introduced through one portal P1 formed in the skin. The tip is shown passing beneath the ligament. The passer is advanced farther until the tip of the passer exits (FIG. 2c) through the other portal P2. Now the end of the wire is released from the tip, and is held while the passer is retracted, FIG. 2d. Once the passer is free of the wrist, it is discarded and only the ends of the wire remain exposed at the site.

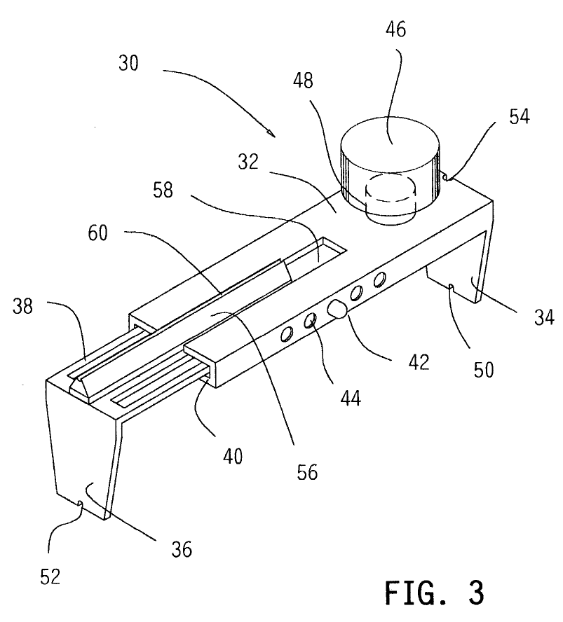

FIG. 3 shows an instrument 30 designed to grip the ends of the wire and tauten it, so that the instrument can be used like a bow saw to cut through the ligament from below. The instrument has a body 32 with a fixed pillar 34 extending perpendicularly downward at one end. A movable pillar 36 is disposed at one end of a slide 38 which is received within a correspondingly shaped cavity 40 in the body. An adjustment pin 42, which is urged outward by a biasing spring, not shown, extends through one of several holes 44 in the side of the body. The length of the instrument can be adjusted by depressing the pin and moving the slide. A rotatable knob 46, fixed to a shaft 48 which serves as a windlass, is disposed at the rear of the body.

The pillars have aligned notches 50, 52 on their bottom edges, to guide the respective ends of the wire as they are brought up around the ends of the instrument. There is also a groove 54 in the rear surface of the fixed pillar, just the top end of the groove being visible in FIG. 3. The rib 56 on the top surface of the slide, situated in the recess 58, has another wire-receiving groove 60 extending along its top apex.

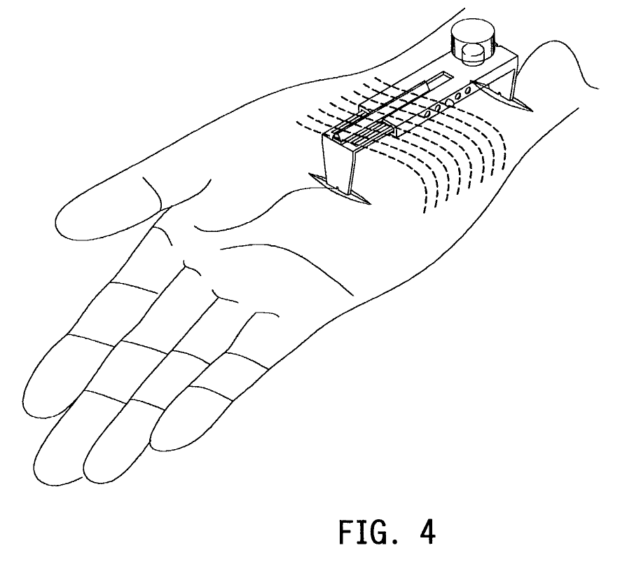

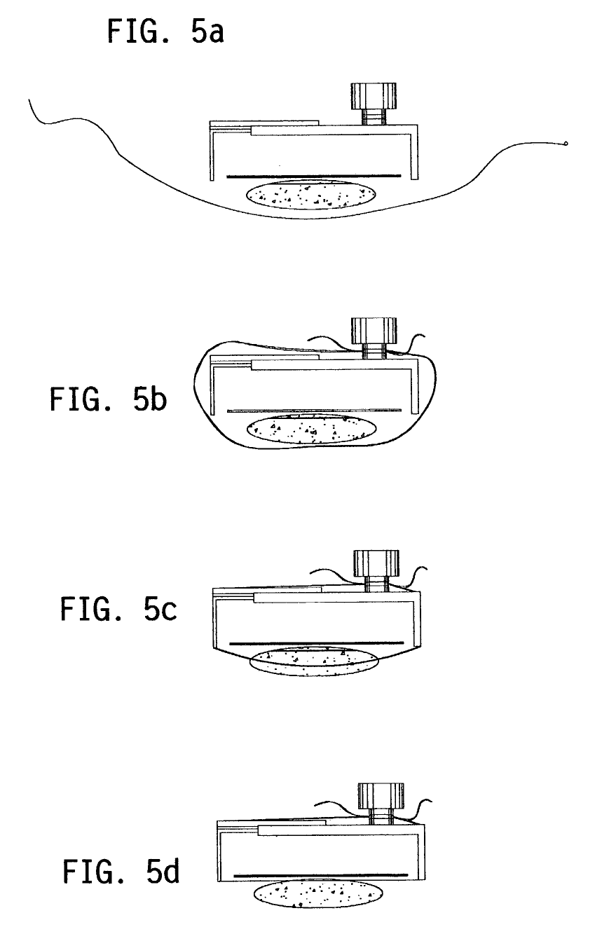

When the instrument is placed on the skin over the site (FIGS. 4 and 5a), the wire ends are passed around the ends of the instrument to the knob, and are wound around the shaft 48, as shown in FIG. 5b. The knob is then turned to tighten the wire, FIG. 5c, and the instrument is moved back and forth to “saw” through the ligament. Alternatively, in some circumstances, a taut small diameter may wire may be able to transect the ligament simply by being pressed against the ligament, that is, a to-and-fro sawing action may not be required. Regardless, once the position of FIG. 5d is reached, the wire is released from the instrument, and may be removed from the site.

There are other ways to secure and tighten the ends of the wire. For example, the knob could be attached to a rack-and-pinion or like mechanism within the body of the instrument which would distend the pillars. In that case, the ends of the wire, rather then being wound on the knob, could be grasped by appropriate clamps or the like on the pillars.

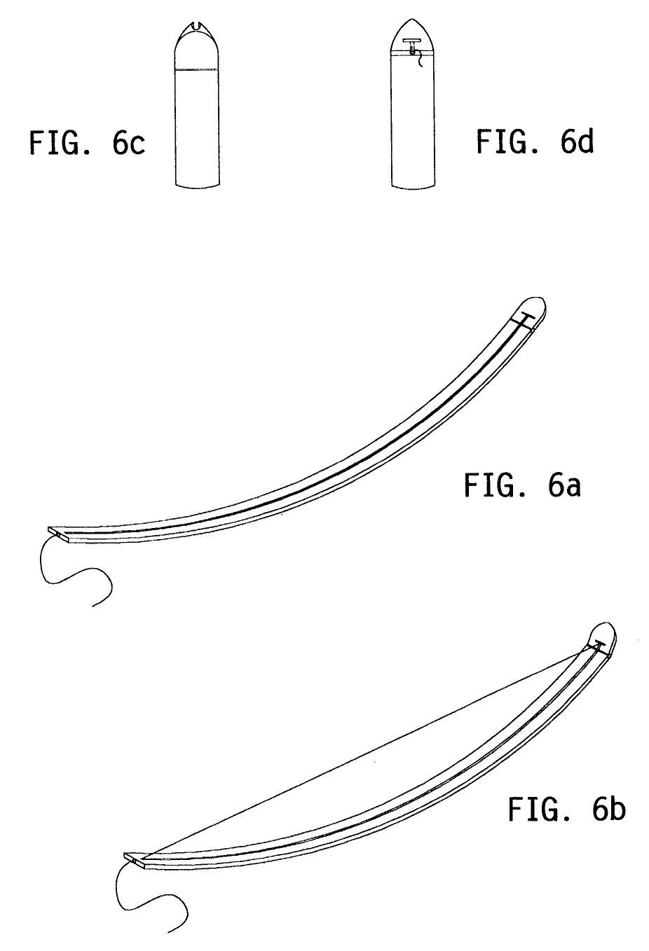

FIGS. 6a-6d show an alternative method, which requires the formation of only a single portal. Here, the distal end of the wire is permanently attached to the tip of the passer. The wire is initially retained (FIG. 6a) within the channel, but can be sprung from the channel to a bow-like configuration (FIG. 6b) by pulling on the proximal end of the wire once the tip of the passer has passed through the carpal tunnel. The tip of the passer may be made so that it can flex, by means of a limited-movement hinge, as shown in FIGS. 6c and 6d. The hinged modification places the wire further away from the body of the passer, so that it is more useful as a saw. Transection is performed, in the single portal method, by reciprocating the passer lengthwise.

Since the invention is subject to modifications and variations, it is intended that the foregoing description and the accompanying drawings shall be interpreted as only illustrative of the invention defined by the following claims.

I claim:

1. A method of transecting the transverse carpal ligament, said method comprising steps of
forming at least one portal in the skin,
inserting a flexible cutting element into the portal and passing it through the carpal tunnel, on one side of the ligament,
drawing the flexible cutting element taut, and then
moving the flexible cutting element against the ligament in such a way as to cut the ligament.

2. The invention of claim 1, wherein the flexible cutting element is a wire.

3. The invention of claim 1, wherein the flexible cutting element is a polymeric filament.

4. A passer for passing a wire through the carpal tunnel beneath the transverse carpal ligament, said passer comprising
a blade sized to pass through the carpal tunnel, said blade having a tapered tip and a channel extending over substantially its entire length for receiving the wire and retaining it in the passer as the passer is passed through the carpal tunnel.

5. The passer of claim 4, in combination with a wire, one end of the wire being permanently affixed to said tapered tip.

6. The passer of claim 5, further comprising a hinge near the tip, for permitting the tip to flex out of the plane of the passer when tension is applied to the wire, to free the wire from the channel so that the passer may be reciprocated lengthwise to transect the ligament.

7. A surgical instrument for tightening a cutting wire which has been passed beneath a ligament during an endoscopic procedure, said instrument comprising
a body having first and second ends,
a rotatable knob, having a shaft, mounted on the body, and
means for guiding opposite ends of the wire around the respective ends of the body to the shaft,
whereby the ends of the wire may be wound around the shaft, so that the wire can be tautened by turning the knob, whereafter the body may be reciprocated lengthwise to cause the wire to cut the ligament.

8. The instrument of claim 7, wherein the body has a fixed pillar at one end and a cavity at the other end, and further comprising a movable pillar having a slide received within the cavity so that the distance between the pillars may be adjusted, and means for maintaining said adjusted distance.

9. The instrument of claim 8, wherein the holding means comprises a spring-loaded pin on the slide which may seat in one of several holes on the body.
